(19) United States
(12) Patent Application Publication
(10) Pub. No.: US 2003/0134271 A1
(43) Pub. Date: Jul. 17, 2003

(54) Diagnosing and monitoring the therapy of stealth virus infections based on the detection of auto-fluorescent material in hair

(76) Inventor: Willam John Martin, South Pasadena, CA (US)

Correspondence Address: W. JOHN MARTIN, 1634 SPRUCE ST., SOUTH PASADENA, CA, 91030, US

(21) Appl. No.: 10/050,232

(22) Filed: Jan. 15, 2002

(51) Int. Cl. C12Q 1/70; G01N 33/53; G01N 33/542

(52) U.S. Cl. 435/5; 435/7.9; 435/7.91

(57) ABSTRACT

A method of presumptive testing of an individual for evidence of infection by a stealth virus that is producing auto-fluorescent materials. The method comprises examination using a fluorescent microscope of hair taken from the individual, for the presence of auto-fluorescence material, similar to that which can be seen in a tealth virus culture obtained from the patient. The level of auto-fluorescence along the shaft of a hair provides a record of the amount of auto-fluorescent material produced over the lifetime of the growing hair. A reduction in the amount of auto-fluorescence can be used to monitor the efficacy of anti-stealth virus therapy.

### CROSS REFERENCE TO RELATED APPLICATIONS

United States Patents

U.S. Pat. No. 5,985,546 Stealth virus detection in the chronic fatigue syndrome William John Martin

U.S. Pat. No. 5,891,468 Stealth virus detection in the chronic fatigue syndrome William John Martin

U.S. Pat. No. 5,753,488 Isolated stealth viruses and related vaccines William John Martin

U.S. Pat. No. 5,703,221 Stealth virus nucleic acids and related methods William John Martin

U.S. Pat. No. 6,022,693 Hair analysis method. Baumgartner

U.S. Pat. No. 5,925,570 Method of measuring metals in samples of living body. Nishidate, et al.

U.S. Pat. No. 5,610,071 Method of hair analysis. Sabal

PCT (Patent Cooperation Treaty)

WO 92/20797 Stealth virus detection in the chronic fatigue syndrome

WO 99/34019 Stealth virus nucleic acids and related methods

WO 99/60101 Stealth Viruses and Related Vaccines

Pending Patent Applications

Therapy of Stealth Virus Associated Cancers and Other Conditions Using Light. Submitted Jan. 7, 2002

Therapy of Stealth Virus Associated Illnesses Using Medium Chain Triglycerides Submitted Jan. 11, 2002

### REFERENCES TO PUBLISHED ARTICLES

Stealth Viruses:

1 Martin W J Chronic fatigue syndrome among physicians. A potential result of occupational exposure to stealth viruses. Explore 2001; 10 (5): 7-10.

2 Martin W J. Stealth Viruses. Explore 2001; 10 (4): 17-19.

3 Durie G M, Collins R. Martin W J. Positive stealth virus cultures in multiple myeloma. A possible explanation for neuropsychiatric co-morbidity. Presented at the Am. Soc. Hematology annual meeting October 2000.

4 Martin W J. Chemokine receptor-related genetic sequences in an African green monkey simian cytomegalovirus-derived stealth virus. Exp Mol Pathol. 2000; 69:10-6.

5 Martin W J. and Anderson D. Stealth virus epidemic in the Mohave Valley: severe vacuolating encephalopathy in a child presenting with a behavioral disorder. Exp Mol Pathol. 1999; 66:19-30.

6 Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.

7 Martin W J. Bacteria-related sequences in a simian cytomegalovirus-derived stealth virus culture. Exp Mol Pathol. 1999; 66:8-14.

8 Martin W J. Stealth adaptation of an African green monkey simian cytomegalovirus. Exp Mol Pathol. 1999; 66:3-7.

9 Martin W J. Cellular sequences in stealth viruses. Pathobiology 1998; 66:53-8.

10 Martin W J. Detection of RNA sequences in cultures of a stealth virus isolated from the cerebrospinal fluid of a health care worker with chronic fatigue syndrome. Case report. Pathobiology. 1997; 65:57-60.

11 Martin W J. and Anderson D. Stealth virus epidemic in the Mohave Valley. I. Initial report of virus isolation. Pathobiology. 1997; 65:51-6.

12 Martin W J. Simian cytomegalovirus-related stealth virus isolated from the cerebrospinal fluid of a patient with bipolar psychosis and acute encephalopathy. Pathobiology. 1996; 64:64-6.

13 Martin W J. Stealth viral encephalopathy: report of a fatal case complicated by cerebral vasculitis. Pathobiology. 1996; 64:59-63.

14 Martin W J. Genetic instability and fragmentation of a stealth viral genome. Pathobiology. 1996; 64:9-17.

15 Martin W J. Severe stealth virus encephalopathy following chronic-fatigue-syndrome-like illness: clinical and histopathological features. Pathobiology. 1996; 64:1-8.

16 Martin W J. Stealth virus isolated from an autistic child. J Autism Dev Disord. 1995; 25:223-4.

17 Gollard R P., Mayr A., Rice D A, Martin W J. Herpesvirus-related sequences in salivary gland tumors. J Exp Clin Cancer Res., 1996; 15: 1-4.

18 Martin W J. and Glass R T. Acute encephalopathy induced in cats with a stealth virus isolated from a patient with chronic fatigue syndrome. Pathobiology. 1995; 63:115-8.

19 Martin W J, et al. African green monkey origin of the a typical cytopathic ‘stealth virus’ isolated from a patient with chronic fatigue syndrome. Clin Diag Virol 1995: 4: 93-103.

20 Martin W J. Stealth viruses as neuropathogens. CAP Today. 1994; 8:67-70.

21 Martin W J. et al. Cytomegalovirus-related sequence in an a typical cytopathic virus repeatedly isolated from a patient with chronic fatigue syndrome. Am J Pathol. 1994; 145:440-51.

### OTHER RELEVANT REFERENCES

Bilinska B. On the structure of human hair melanins from an infrared spectroscopy analysis of their interactions with Cu2+ ions. Spectrochim Acta A Mol Biomol Spectrosc 2001;57:2525-33

Garcia-Rivera J, Casadevall A. Melanization of Cryptococcus neoformans reduces its susceptibility to the antimicrobial effects of silver nitrate. Med Mycol 2001;39:353-7

Kayatz P, et al. Oxidation causes melanin fluorescence. Invest Ophthalmol Vis Sci January 2001; 42(1):241-6

Suzuki et al., Forensic Sci. International, 24:9-16, 1984.

Tobin D J, Paus R. Graying: gerontobiology of the hair follicle pigmentary unit. Exp Gerontol January 2001;36(1):29-54.

Tsuchida M. et al. Lipofuscin and lipofuscin-like substances. Chem Phys Lipids 1987;44:297-325

### STATEMENT REGARDING FEDERALLY SPONSORED RESEARCH OR DEVELOPMENT

No Federal funding was received in support of the research covered in this patent application.

### REFERENCE TO SEQUENCE LISTING, A TABLE, OR A COMPUTER PROGRAM LISTING COMPACT DISK APPENDIX

None provided.

### BACKGROUND OF THE INVENTION

The present invention relates to the diagnosis and to the monitoring of therapy of virus infections, in which the virus belongs to a group of a typically structured, non-inflammation-inducing viruses, for which the inventor has coined the term stealth viruses. The patent application relates particularly to the detection of stealth virus induced auto-fluorescent material in hair and other locations in patients infected with a stealth virus. Methods for the detection and characterization of stealth viruses are covered in U.S. Pat. Nos. 5,985,546; 5,891,468; 5,753,488; and 5,703,221. Although initially identified in association with neuropsychiatric illnesses, including the chronic fatigue syndrome, stealth viruses can also commonly be cultured from cancer patients. (Stealth virus-related references are listed in this application and are numbered 1-21. All cited patents and entire list of stealth virus publications, are incorporated herein by reference).

The basis of the present invention is the discovery that certain stealth viruses lead to marked intracellular and subsequent extra-cellular accumulations of a diverse range of particulate materials, some of which are photo-(light) sensitive. Light is the visible portion of the extremely broad spectrum of electromagnetic radiation, that also includes non-visible energies such as gamma rays, X-rays, microwaves, radio-waves, and both ultraviolet and infrared radiations. Light is best understood in terms of energy packets termed photons, that travel through space in an oscillating sine-wave form. The energy of each photon is determined by the frequency of the fluctuating wave, which is typically measured as a wavelength of each full oscillation. Visible white light comprises a mixed range of colors that extend from the relatively short wavelengths around 400×10⁻⁹ meters, (400 nanometers) for purple light to the longer wavelengths of around 600-700 nanometers (nm) for the varying shades of red light. Portions of the light spectrum of white light are either absorbed or reflected by all but fully transparent objects. The perceived color of an object is that of the reflected light waves. For the vast majority of objects, the energy of the absorbed light is dissipated as an increase in the motion of the individual molecules that comprise the object. This results in a rise in the temperature of the object. For certain compounds, however, parts of the energy of the incoming light can cause an outward orbital shift in certain electrons surrounding individual atoms. As these “excited” electrons return towards their previous energy levels, they can release photons and become a source of re-emitted light. The electrons can also become involved in mediating certain chemical reactions and ion transfers that are dependent upon, and driven by, the heightened energy state.

The term “photosensitive” specifically refers to the ability of certain materials to absorb energy from light, at one or more particular wavelengths, and to subsequently emit some of the absorbed energy as light, usually at a longer wavelength than that causing the photo-excitation. This light absorption/re-emission process is termed auto-fluorescence. It is generally known that unregulated and/or excessive light induced fluorescence occurring within a cell can have detrimental effects on cell vitality and can lead to cell death.

Stealth viruses can be cultured from blood and other tissues of stealth virus infected patients. Long term stealth virus cultures will commonly show the formation of auto-fluorescent materials. I have shown that cell damage can be induced in stealth virus infected cells by using light. As disclosed in the co-pending patent application “Therapy of Stealth Virus Associated Cancers and Other Conditions Using Light,” the administration of tissue penetrating light can potentially be used to kill stealth virus infected cancer cells. The light excitation/light emission wavelengths of stealth virus induced auto-fluorescent materials, can vary between different cultures. In working towards the wider application of light therapy to multiple cancer patients, I had planned to culture the actual stealth virus infecting each individual patient. This would allow me to specifically test each culture for the production of auto-fluorescent materials and to define the optimal, tissue penetrating light wavelengths, that would cause maximum cell damage. The present invention provides an alternative approach to the detection and characterization of the auto-fluorescent material being produced by the stealth virus infecting a patient. In addition, it provides a method to assess the ability of various other forms of anti-stealth virus therapy to lead to a body-wide reduction in the production of stealth virus induced, auto-fluorescent materials.

### BRIEF DESCRIPTION OF THE DRAWINGS (FIGURES)

The accompanying figures, which are incorporated in and constitute a part of the specification, illustrate the auto-fluorescent thread-like structures seen in stealth virus cultures. They also illustrate the auto-fluorescence seen with hair samples obtained from a stealth virus infected patient. Additional informative figures are also available in the co-pending patent applications: “Therapy of Stealth Virus Associated Cancers and Other Conditions Using Light,” and “Therapy of Stealth Virus Associated Illnesses Using Medium Chain Triglycerides.” Taken together, the figures serve to explain the foundations and the principles of the invention.

FIG. 1. A ribbon-like structure extending from a cluster of cells in a positive stealth virus culture. Relatively normal appearing fibroblasts can be seen in the background. This photomicrograph was taken at low power (10× objective) using phase contrast bright field illumination.

FIG. 2. Dark field illumination of a freely floating thread-like strand of material seen in a stealth virus culture.

FIG. 3. Dark field illumination of a multi-colored (mainly blue) appearing long thread-like structure seen as a twisted knot, under low power (10× objective) in a stealth virus culture

FIG. 4. Two views of a continuous, very long twisted and knotted thread-like structure, seen in a stealth virus culture using dark field illumination.

FIG. 5. Two views of a continuous, very long twisted and knotted thread-like structure, seen in a different stealth virus culture than that shown in FIG. 4.

FIG. 6. Transmission photomicrograph of a hair taken from a normal individual.

FIG. 7. A white-on-black depiction of the faint green auto-fluorescence that was seen when illuminating the hair shown in Figure with an argon laser (blue light at 488 nm). No auto-fluorescence was seen when the same hair was illuminated with a helium/neon laser (green light at 543 nm).

FIG. 8. Transmission photomicrograph of a hair taken from the stealth virus culture positive patient.

FIG. 9. A white-on-black depiction of the bright, diffuse green auto-fluorescence seen when illuminating the hair shown in Figure with an argon laser (blue light at 488 nm).

FIG. 10. A white-on-black depiction of the very bright and narrowly restricted red auto-fluorescence seen when illuminating the hair shown in figure with a helium/neon laser (green light at 543 nm).

### BRIEF SUMMARY OF THE INVENTION

A method of presumptive diagnosis and also of monitoring the efficacy of therapy in a stealth virus infected patient comprising the examination of a hair sample of the patient for the presence of auto-fluorescent material, similar to that found, or expected to be found, in a stealth virus culture of the blood of that patient. The method disclosed in this invention stemmed from an earlier discovery that stealth virus infected cultures will commonly produce abnormal, aggregated, intracellular and extra-cellular materials, and that some of this material will auto-fluorescence when exposed to light of certain wavelengths. Included in the extra-cellular, auto-fluorescent materials, can be long thread-like structures that develop in certain stealth virus cultures that are maintained for several weeks. I had reasoned that these thread-like structures, reflected an attempt by the infected cells to externalize some of their potentially toxic, auto-fluorescent materials.

Upon reviewing the appearance of these thread-like structures, I realized that in many ways they were reminiscent of a growing hair. They even tended to become knotted over time. The generally accepted functions of hair are to provide body warmth and some protection from the sun's ultra-violet light. Hair is pigmented because it contains melanin. Various metals will bind to melanin and elevated levels of specific metals can be detected in hair following exposure to toxic levels of the corresponding metal (Bilinska B. On the structure of human hair melanins from an infrared spectroscopy analysis of their interactions with Cu2+ ions. Spectrochim Acta A Mol Biomol Spectrosc 2001;57:2525-33). In this respect, the body's hair is providing an excretory pathway to help diminish the burden of a toxic metal overload. I reasoned that hair could also be providing an excretory mechanism to help remove toxic auto-fluorescent molecules produced by stealth virus infections. I, therefore, examined body hairs from a stealth virus infected patient. Using fluorescence microscopy, I observed green and red auto-fluorescence in the hair sample. The red auto-fluorescence was strikingly similar to the auto-fluorescence I had seen in the stealth virus culture from this particular patient. As expected, no red fluorescence, and only very minimal green fluorescence was seen in the hair samples from non-infected individuals. I, thereby, realized that I could use hair auto-fluorescence, as a presumptive, surrogate diagnostic marker for the presence of a long standing stealth virus infection in a human or animal patient. More importantly, I realized that the reduction in the level of hair auto-fluorescence in newly growing hair, could provide a method to monitor therapy that was being given to suppress a stealth virus infection.

### DETAILED DESCRIPTION OF THE INVENTION

The present invention provides a method to presumptively diagnose, and to monitor the efficacy of therapy, of stealth virus infections occurring in a human or animal subject. The method is based on determining the presence of auto-fluorescent material in body hairs taken from the human or animal subject. The auto-fluorescent material can be directly visualized using a fluorescent microscope or, alternatively, the hair, or material extracted from the hair can be analyzed for fluorescence using spectroscopy. The optimal excitation frequency of light required to evoke maximum fluorescence from a sample of body hair, and the actual frequencies of the emitted fluorescent light, can be determined. These values can be directly compared with those obtained in the examination of stealth virus cultures derived using a blood sample obtained from the particular patient. Identity of the values can provide confirmation that the auto-fluorescent material in the hair is derived from stealth virus infected cells, or possibly also from stealth virus infected bacteria, in the patient. The relative intensity of fluorescence along the length of a single hair can provide an indication of changes in stealth virus disease activity over time. More importantly, diminishing amounts of auto-fluorescent material in newly growing hair can be taken as a presumptive sign of lessening viral activity within an individual undergoing anti-stealth virus therapy.

Although not commonly performed in mainstream medicine, hair analysis has been used by many physicians practicing alternative medicine. Two large diagnostic laboratories in the United States specialize in performing hair analysis. The laboratories are Great Smokies Diagnostic Laboratory, and Doctors' Data Laboratory. The primary use of hair analysis has been to identify presumptive toxic exposure to various heavy metals such as lead, mercury, cooper and zinc. High levels of any of these metals have been used to justify the administration of metal-binding (chelating) agents in what is known as detoxification therapy. In the case of high mercury levels, certain physicians will also recommend the removal of all mercury containing dental amalgams.

Metal poisoning is commonly regarded by many alternative health practitioners as a major cause of chronic debilitating illness. It is not surprising, therefore, that among the patients referred for stealth virus testing, a number have had hair analysis performed. From reviewing many of these laboratory reports, I have frequently noted elevations in one or more of the routinely tested metals, such as aluminum, antimony, arsenic, barium, bismuth, cadmium, lead, mercury, nickel, tin, and uranium. There has been no consistent pattern, except for a more common increase in mercury compared to the other elements.

An understanding of the role of metal ions in normal cell biology is still incomplete. It is known that the function of certain proteins are dependent on having one or more metal ions attached to the protein in either covalent or ionic linkage. Prominent examples include the role of iron in hemoglobin, and zinc in various enzymes. Metals can also play an important part in the electron and energy transfers that can accompany fluorescence. For example, manganese is an essential component of chlorophyll, while iron is part of the highly fluorescent protoporphoryn IX molecule. The attraction of fluorescent molecules to certain metals may be governed, in part, by the relative ease of electron transfers to and from metal atoms. Excess amounts of metals can interfere with the normal functions of certain proteins and can thereby exert toxic, inhibitory effects.

Human hair is a rich source of a family of pigmented proteins known as melanins. Hair color is determined by the amount and type of melanin present in the keratinocytes (fully differentiated squamous epithelial cells), that constitute the root and shaft of the hair fiber. Melanin is known to bind to various metals and, thereby, to act as a chelating agent. Melanin-like pigments may have a role in providing protection of fungi and plants from metal toxicity by also acting as a chelating agent (Garcia-Rivera J, Casadevall A. Melanization of Cryptococcus neoformans reduces its susceptibility to the antimicrobial effects of silver nitrate. Med Mycol 2001;39:353-7). While not widely considered as a disposal mechanism in animals, melanin-containing hair could help rid the body of potentially toxic materials that would bind to melanin or to keratin. This concept has been expressed in at least one published article (Tobin D J, Paus R. Graying: gerontobiology of the hair follicle pigmentary unit. Exp Gerontol 2001; 36: 29-54).

Stealth virus cultures will typically show the production of both intracellular and extra-cellular particulate, pigmented materials. Similar appearing intra- and extra-cellular materials can also be seen in tissue biopsies from stealth virus infected patients and animals, especially when using periodic acid Schiff (PAS) staining. By examining stealth virus infected cell cultures over time, I have frequently observed clusters of infected cells sequestering (localizing) the intracellular pigmented material. Some of the materials can actually be discharged from the cell clusters. I have also observed the formation of long thread-like, tubular and ribbon shaped structures, growing out from cell clusters in stealth virus cultures. These structures will commonly show auto-fluorescence and may reflect an attempt by the infected cells to externalize potentially toxic, auto-fluorescent materials. The thread-like tubular structures are very reminiscent of a growing hair. They even become knotted over time. I, therefore, extrapolated that what was seemingly occurring in the cultures, could be occurring throughout the body. In other words, the body could be using its growing hair as a pathway to try to rid itself of the harmful auto-fluorescent material being produced in the body by stealth virus infected cells.

If this were to be so, then body hair from a stealth virus infected patient should contain auto-fluorescent material, similar to that seen in the stealth virus cultures from that patient. I initially tested this proposition using a hair sample from a patient whose blood samples I knew had produced brightly red auto-fluorescent material. I used a microscope with matching blue, green and red filters, which were placed in the light path, below and above the sample stage. Using this approach, I readily detected green and red auto-fluorescence in the hair sample. I next examined a mixture of hair collected from a local barber shop floor. The majority of the hairs examined showed little or no evidence of any green auto-fluorescence, and absolutely no red auto-fluorescence was seen. I confirmed the strong green and red auto-fluorescence of hairs from the stealth virus infected patient using a Zeiss confocal microscope with both mercury lamp and laser illuminations. As illustrated below, the hair sample from the known stealth virus positive patient, gave a striking, uniform narrow band of strong red auto-fluorescence when illuminated with the green laser light (543 nm wavelength). It also showed a strong and more diffuse green fluorescence when illuminated with a blue laser light (488 nm). Among several hairs from normal individuals, only a very faint green fluorescence was seen. A hair sample from the stealth virus infected patient was also tested for its heavy metal content at the Great Smokies Diagnostic Laboratory (Ashville, N.C.). As shown below, it had elevated levels of various metals, including mercury.

This particular individual is currently being treated for his stealth virus infection using transdermally administrated medium chain triglyceride, along with other supplements. The adequacy of the triglyceride therapy is being monitored by showing stealth virus inhibitory activity in chloroform extracted urine, from which the chloroform is being removed by heat evaporation. Evidence for a reduction in hair associated auto-fluorescence will be sought at weekly intervals by examining hair from shaving. Serial studies of hair auto-fluorescence will also be conducted along the length of a body hair plucked from a non-sun exposed area, as for example a pubic hair. These and similar studies will have wide applications to the management of stealth virus infected patients.

As used herein stealth or stealth-adapted viruses refers to infectious agents that will induce a characteristic vacuolating cytopathic effect (CPE) in human and animal tissue culture cells using procedures described for the cultivation of stealth viruses. These procedures have been provided in various patents and publications relating to stealth viruses. Essentially, it is possible to demonstrate the presence of a stealth virus in peripheral blood or tissues of a stealth virus infected patient, by following tissue culture procedures that will allow for the expression of a stealth virus induced CPE. A suitable procedure is as follows: Mononuclear cells are separated from 8 mls of whole blood, collected in an acid citrate dextrose (ACD) blood vacutainer tube using Ficoll Paque (Pharmacia, N.J.) density centrifugation. After washing the mononuclear cells in phosphate buffered saline, they are re-suspended in 2 ml of serum free, X Vivo-15 medium (BioWhittaker Inc., MD). The cells are aliquoted into two vials, each of which is stored frozen until testing. The supernatant and the cell pellet from a lightly centrifuged thawed vial are each added to culture test tubes containing MRC-5 human fibroblasts (BioWhittaker Inc., MD), in 3 ml of serum free X Vivo-15 medium. The culture tubes are placed on a slowly rotating wheel (Cel-Gro, Lab Line, Medford Ill., 4 minutes per rotation) in a 36.5° incubator. The tubes are examined regularly using an inverted phase contrast microscope. The appearance, rate of progression and host range of the CPE caused by stealth-adapted viruses are quite dissimilar from those caused by any of the commonly encountered conventional human cytopathic viruses, including human herpes simplex viruses, cytomegalovirus, Epstein-Barr virus, varicella-zoster virus, human adenoviruses, measles virus, or enteroviruses. In the case of MRC-5 human fibroblast indicator cells, the normal spindle shaped, translucent, closely packed cells become enlarged, rounded and tend to fuse into small, and later into larger, three dimensional cell syncytia and clusters. The cellular cytoplasm displays a vacuolated, lipid-laden-like appearance. With time, and especially in larger cell clusters, an additional accumulation of yellow-brown to golden-black, fine and/or coarse pigmentation, can be readily seen within affected cells, and sometimes in the culture supernatant. Even more striking is the formation of long extra-cellular pigmented thread, ribbon and tube-like structures in many of the longer-term cultures. These unusual structures are not seen in cultures of conventional cytopathic viruses. Many of these structures can be shown to be auto-fluorescent. While there are major overall similarities between stealth virus cultures from different patients, there are also many subtle differences, especially in terms of the extent, coarseness, color, types and auto-fluorescent characteristics of particulate intracellular and extra-cellular materials, and in the tendency to form smaller or larger cell syncytia and cell clusters.

Because they can capture, amplify and mutate genes of viral, cellular and bacterial origins, stealth viruses are easily misidentified as various types of conventional viral and bacterial pathogens. Positive stealth viral cultures are commonly found in patients diagnosed by their own clinicians as having chronic Lyme disease, chronic mycoplasma infection, and human herpesvirus-6 infections. These clinical diagnoses are typically based on the results of assay systems that do not exclude false positive results resulting from the presence of a stealth virus infection. This specification is intended to include all patients in whom it could be shown that they would give a positive stealth virus blood culture using, for example the method described above, and in whom the culture would show the production of auto-fluorescent material. The specification is also intended to include patients in whom the detection of auto-fluorescent material in a hair sample is being determined as a surrogate, presumptive, diagnosis for the in vivo production of auto-fluorescent material of an infectious viral or bacterial origin. In this regard, apart from our finding with stealth viruses, no commonly identified human viral or bacterial pathogen has been associated with the production of auto-fluorescent materials.

The auto-fluorescent materials in the hair can be relatively easily fractionated from non-fluorescent material using methods well known to biochemists. The metal content of the auto-fluorescent material can be determined using atomic spectroscopy on purified, auto-fluorescent material. If a close association is found, it would suggest that the auto-fluorescent material is simply trapping the metal, rather than there being a toxic over-supply of the metal throughout the body.

While the currently preferred method for detection of auto-fluorescence within hair comprises taking a few strands of hair for fluorescent microscopic analysis, other methods can be easily envisioned. These could include, but are not limited to; i) exposing hair while it is still on the body to a light source set at a particular wavelength and capturing any light that is emitted at a longer wavelength; ii) using an energy source, other than light, to activate light emission from the auto-fluorescent material present in the hair of a stealth virus infected patient. These sources can include radio-frequencies, electric and magnetic fields, and ultra-sound; and iii) using either fingernails or toenails, rather than hair for analysis (Suzuki et al., Forensic Sci. International, 24:9-16, 1984).

The potentially harmful effects that auto-fluorescence has on hair vitality is not being addressed in this specification. It is clear, however, that sunlight and other light sources that are inducing hair auto-fluorescence, could have damaging effects on both T hair and skin. Various substances can be used to absorb (quence) fluorescence. The use of such substances could conceivably be of benefit to patients with auto-fluorescent hair.

### EXAMPLES AND ILLUSTRATIONS

Examples of positive stealth virus cultures are provided in co-pending applications “Therapy of Stealth Virus Associated Cancers and Other Conditions Using Light,” and “Therapy of Stealth Virus Associated Illnesses Using Medium Chain Triglycerides.” These applications also contain several illustrations of the thread-like structures seen in long-term stealth virus cultures of certain patients. Additional examples are provided herein.

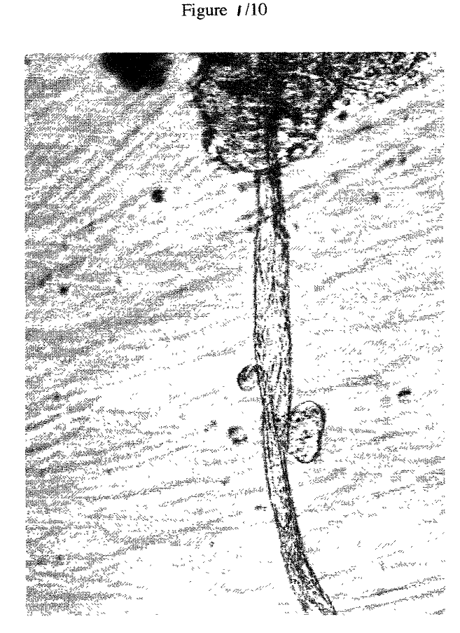

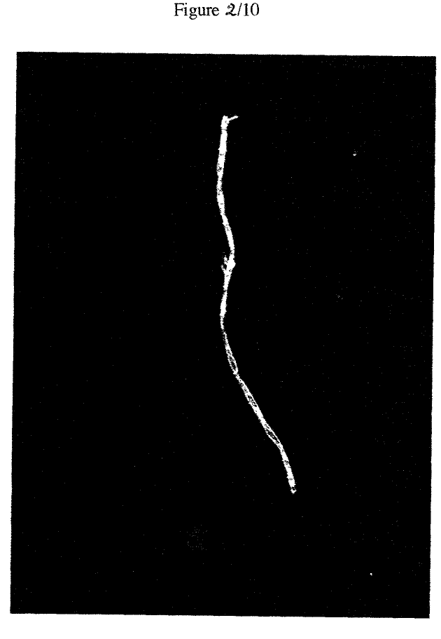

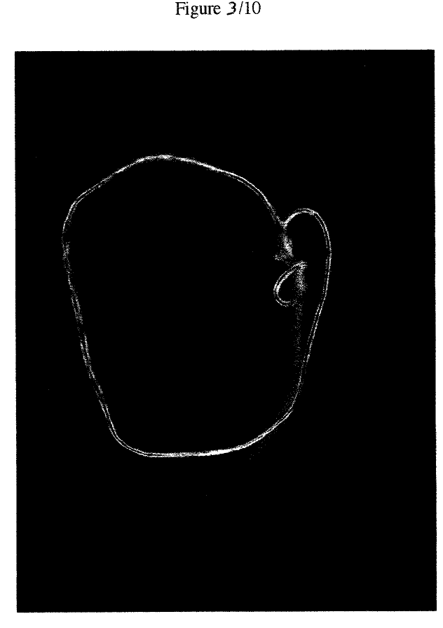

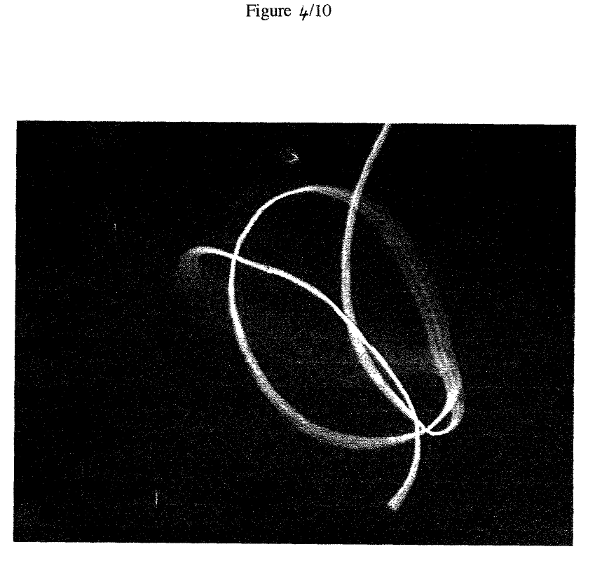

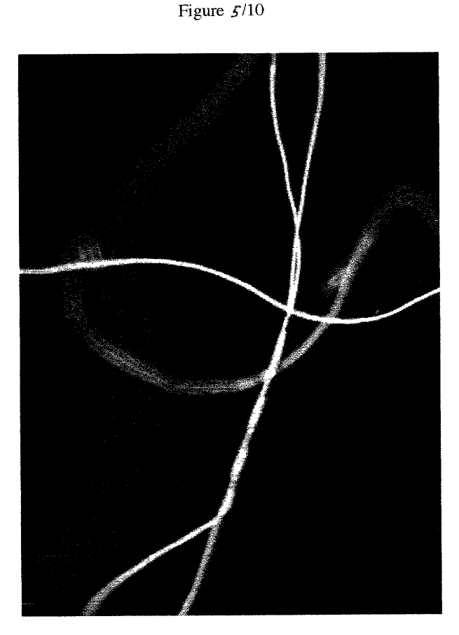

The preferred method of promoting the formation of ribbon and thread like structures from a stealth virus culture, is to refrain from re-feeding the culture after a strong CPE has developed. Cells in the cultures will generally stay viable for 1-2 months, during which time, there can be significant re-growth of relatively normal appearing cells. There can also be the formation of thread-like and other structures, that are initially attached and growing from a focus of cells. FIG. 1, shows a fairly typical long ribbon-like thread that grew out from a cluster of cells. the ribbon shows a definite interwoven pattern. The threads tend to become detached and to subsequently float freely in the tissue culture medium. They can show varying colors under both phase contrast and dark field illumination. FIG. 2, shows a black and white, dark field photomicrograph of a floating tube-like strand. FIG. 3 is from a different culture. This knotted, thin walled, tube like structure, showed a variable, refractile, blue coloration. FIGS. 4 and 5 are included to emphasize the length to which some of these thread-like structures can grow. In neither culture, was it possible to capture all of the twisted and knotted floating thread-like strand in a single low power photomicrograph.

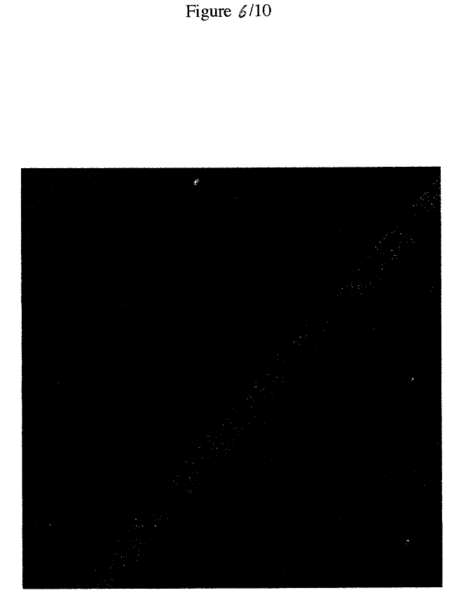

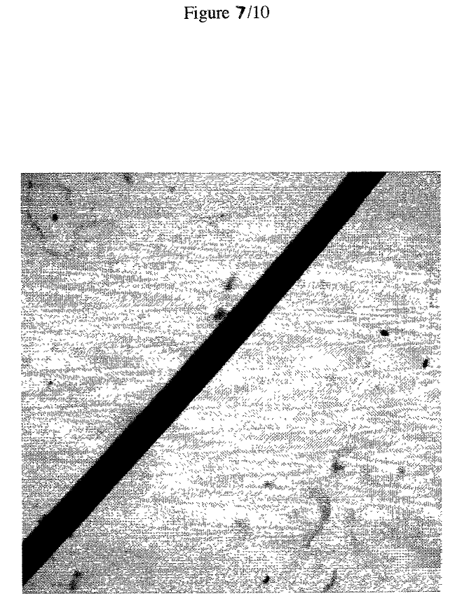

The next series of photographs were taken using a Zeiss confocal microscope. FIG. 6, is a transmission photomicrograph of a hair from a healthy individual. The hair showed no obvious fluorescence when illuminated with white light provided by the mercury lamp of the microscope. There was, however, discernable, low level emission of green auto-fluorescence upon exposure to blue laser light from an argon lamp (488 nm wavelenght excitation). This is seen in FIG. 7, where the white dots reflect light passing through a green filter. Absolutely no auto-fluorescence was seen using 543 nm helium/neon laser green light excitation, and passing the image through a red filter.

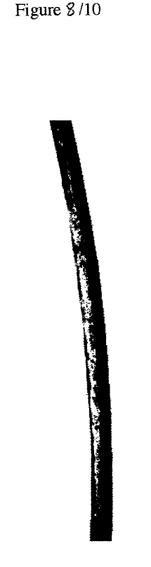

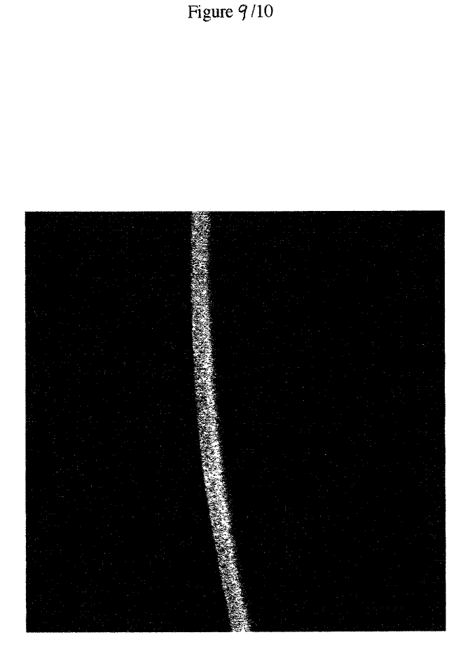

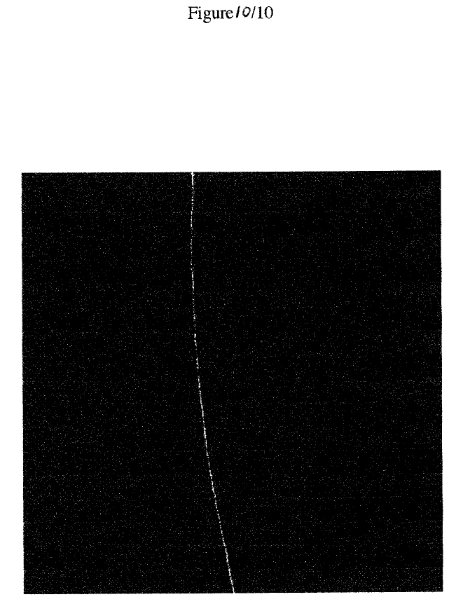

FIG. 8 is a transmission photomicrograph of a hair from the stealth virus infected patient. It showed both green and red fluorescence when illuminated with white light from the mercury lamp. Using the blue laser, considerably more green fluorescence was seen diffusely along the hair strand (FIG. 9). More striking, was the very bright, but narrow band of red auto-fluorescence elicited by exposure of the hair to 543 nm helium/neon laser green light excitation, and passing the image through a red filter (FIG. 10). Red auto-fluorescence is uncommonly seen in biological samples. Yellow auto-fluorescence can be seen with oxidized melanin (Kayatz P. Invest Ophthalmol Vis Sci 2001;42:241-6) and with certain lipofuscins (Tsuchida M. et al. Lipofuscin and lipofuscin-like substances. Chem Phys Lipids 1987;44:297-325). It is, however, a feature of materials present in a number of the stealth virus cultures examined, and is particularly impressive in cultures from the patient whose hair sample is shown in FIGS. 8-10.

Hair Analysis for Chemicals:

The following Table lists the results of testing a hair sample for metals and other components. The hair was obtained from the stealth virus infected patient whose hair sample was used in the above auto-fluorescence experiment.

Hair Analysis*

| | Concentration | Normal Range | |
|---|---|---|---|
| Element | Parts per million | Parts per million | Interpretation |
| Mercury | 6.73 | 0.00-1.00 | Extremely high |
| Strontium | 13.85 | 0.35-3.25 | Extremely high |
| Calcium | 3,898 | 220-780 | Extremely high |
| Magnesium | 239 | 16-90 | High |
| Zinc | 257 | 120-170 | High |
| Iron | 22.9 | 6.0-18.0 | Slightly elevated |
| Cobalt | 0.0574 | 0.0075-0.0400 | Slightly elevated |
| Lithium | 0.0845 | 0.0027-0.0320 | Slightly elevated |

The following elements were found to be in the normal range: Aluminum, Antimony, Arsenic, Barium, Bismuth, Boron, Cadmium, Chromium, Cooper, Lead, Manganese, Molybdenum, Nickel, Rubidium, Selenium, Sulfur, Thallium, Tin, Uranium, and Vanadium. * Testing performed by Great Smokies Diagnostic laboratory, Ashville, N.C., 28801. The interpretation was included in the laboratory report.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention

What I claim as my invention is:

1. A method to test for t he ability of a stealth virus infection to induce the production of auto-fluorescent materials in a human or animal subject, comprising collecting one or more body hairs from the subject, illuminating the hairs with light of known wavelengths, and examining the illuminated hairs, using either a fluorescent microscope or a spectrophotometer, for the emission of light at a wavelength different from that used to illuminate the hairs.

2. A method to test for variations in the levels of production of auto-fluorescent materials over time in a stealth virus infected patient by comparing the intensity pattern of the auto-fluorescence seen along the length of a hair fiber, and/or by comparing the intensity of auto-fluorescence seen in hair samples taken at different times during the course of a stealth virus associated illness.

3. The method of claim 2 in which the testing is performed as a way to monitor the effectiveness of anti-stealth virus therapy directed at suppressing stealth virus activity in an infected patient, as shown by a reduction in the intensity of auto-fluorescence in the more recently formed portion of the hair.

4. A method to test for the production of auto-fluorescent materials in a human or animal subject, comprising collecting one or more body hairs from the subject, illuminating the hairs with light of known wavelengths, and examining the illuminated hairs using either a fluorescent microscope or a spectrophotometer for the emission of light at a wavelength different from that used to illuminate the hairs.

5. The method of claim 4 in which the testing is performed as a way to monitor the effectiveness of therapy directed at suppressing the production of auto-fluorescent materials in a human or animal subject.

6. The method of claim 4 in which the hairs are collected from a part of the body that is not normally exposed to sunlight, such as pubic hairs and underarm hairs.

7. The method of claim 4 in which the hairs are collected from freshly shaven beard growth.

8. A method to test for the production of auto-fluorescent materials in a human or animal subject, comprising collecting fingernails or toenails from the subject, illuminating the nails with light of known wavelengths, and examining the illuminated hairs using either a fluorescent microscope or a spectrophotometer for the emission of light at a wavelength different from that used to illuminate the hairs.

9. A method to test for the ability of a stealth virus infection to induce the production of materials in a human or animal subject that can be activated by an energy source other than light, comprising collecting one or more body hairs from the subject, exposing the hairs to the energy source, and examining the energy-exposed hairs for light emission by using a photographic film.

10. The method of claim 9, in which the energy source is provided by any of the following modalities used alone or in combination with one another: radio-frequency radiation, electric field, magnetic field, or ultrasound vibration.
